(19) United States
(12) Patent Application Publication
(10) Pub. No.: US 2005/0220857 A1
(43) Pub. Date: Oct. 6, 2005

(54) Physiologically compatible, phospholipid-containing, stable and hard matrix

(76) Inventors: Martin Purpura, Bonn (DE); Dirk Cremer, Zolling (DE); Ralf Jager, Freising (DE); Michaela Thalhammer, Freising (DE)

Correspondence Address: FITCH EVEN TABIN AND FLANNERY, 120 SOUTH LA SALLE STREET, SUITE 1600, CHICAGO, IL, 60603-3406, US

(21) Appl. No.: 10/511,884

(22) PCT Filed: Apr. 17, 2003
(86) PCT No.: PCT/EP03/04031

§ 371 Date: May 4, 2005

(30) Foreign Application Priority Data
Apr. 19, 2002 (DE) 102175551

(51) Int. Cl. A61K 9/127

(52) U.S. Cl. 424/450

(57) ABSTRACT

The invention concerns a physiologically compatible, phospholipid-containing, stable and hard matrix consisting of a supporting material and a bioactive component which has a total diameter of between 0.1 and 5000 μm and contains ≧5% by weight, based on the starting material, of acetone-insoluble components as the bioactive component, which are in particular phosphatidyl serine, phosphatidyl choline and such like, as well as lyso variants and/or derivatives thereof. This matrix which preferably has a supporting material consisting of carbohydrates and/or proteins and preferably additional bioactive substances as ingredients such as amino acids, vitamins, trace elements and similar substances, can have liquid contents and is used in particular in functional foods, special foods and dietary supplements.

The present invention concerns a physiologically compatible, phospholipid-containing, stable and hard matrix in particular a microcapsule consisting of a supporting material and a bioactive component.

The phospholipid class of substances are so-called complex lipids having amphiphilic properties i.e. they are at the same time lipophilic and hydrophilic which, among others, enables them to form lipid bilayers in aqueous media.

Phospholipids (also referred to as phosphatides) are chemically phosphodiesters in which the phosphoric acid is esterified, on the one hand, with a sphingosine or glyceride residue and, on the other hand, with choline, ethanolamine, serine, inositol or glycerol. Phosphatidyl choline is also known as lecithin and at the same time is an eponym for a large group of special phospholipids, the lecithins. Phosphatidyl serine and phosphatidyl ethanolamine are also referred to as cephalins.

The lyso derivatives which also belong to this group are formed by hydrolytic cleavage with specific phospholipases.

Phospholipids are typically insoluble in acetone which is why they are also referred to as acetone-insoluble phosphatides or acetone-insoluble substances. Lecithins are mixtures or fractions of phosphatides which are isolated by physical processes from animal or vegetable foods; lecithins contain at least 60% substances that are insoluble in acetone. Due to this property lecithin-containing products can be tested for their actual phosphatide or phospolipid content with the aid of the so-called acetone solubility test.

Phospholipid-containing capsules are well-known from the prior art and contain phospholipids usually as a coating substance. If phospholipids are used in the filling, i.e. in the core of the capsule, they usually act there in small amounts as a formulation adjuvant usually having solubilizing properties.

As a result of their amphiphilic properties phospholipids are also used as coating substances in the known liposomes and transferosomes. In this connection they are used especially in the field of mucosal applications due to their bioadhesive properties where they are introduced in particular into nasal and oral cavities.

However, in a chemically-modified form phospholipids are also used as surface-active formulation adjuvants (surfactants).

It is also known that vesicles which carry phospholipids as a coat can be produced by ultrasound.

Special granulates with lecithin coats are known from the Japanese Application JP 91 47 043 and from EP-A 493 441. These granulates which, among others, contain steroids as bioactive substances are used as animal feed additives.

According to WO 87/04347 lyso-phospholipids are described as solubilizers for hydrophobic bioactive substances.

Forms of administration that can enter the lungs which use organic halogen compounds as a carrier for the phosphatidyl choline are described in the International Applications WO 99/16419 and 99/16421.

Soft gelatin capsules containing lecithin as a bioactive ingredient are commercially available as KAL®-Lecithin and contain 1200 mg soybean lecithin. However, in order to accommodate this amount of lecithin in a capsule, capsule sizes have to be selected which come close to the centimetre limit and thus give rise to a limited compliance.

A process for producing phosphatidyl serine (PS), i.e. a phospholipid, is known from the German Patent DE 199 17 249. In this connection it is stated that PS or corresponding PS products obtained in this manner can be stabilized in aqueous systems by embedding them in a hard fat. However, the statements made in this patent are limited to soft gelatin capsules which should have the special PS in their contents.

Hence based on the prior art the object of the present invention was to provide a physiologically compatible, phospholipid-containing, stable and hard matrix composed of a supporting material and a bioactive component containing, on the one hand, phospholipid fractions relative to the starting material in fractions that exceed the known amounts in which phospholipids are used as coatings or formulation agents, and thus contain amounts of phospholipids that can for example serve as food supplements. However, on the other hand, the matrix should have a size that allows it to be ingested easily and problem-free, but is at the same sufficient to take up an adequate amount of phospholipid to achieve a physiological effect.

This object was achieved by a corresponding matrix which has a total diameter between 0.1 and 5000 μm and contains ≧5% by weight, preferably ≧10% by weight and especially ≧20% by weight, based on the starting material, of acetone insoluble phosl)holipid components as the bioactive component.

Surprisingly it has turned out that this matrix according to the invention significantly increases compliance in accordance with the object of the invention since the small size of the matrix simplifies intake and especially does not negatively influence the swallowing sensation. In addition the matrix according to the invention can contain amounts of phospholipid with a bioactive effect that enable a better dosing of the daily amount. It was also not to be expected that the matrix and above all the phospholipid component would have a significantly higher stability towards destructive influences such is humidity, light and temperature, i.e. in general oxidative and/or hydrolytic effects. Furthermore, the bioavailability of the phospholipids administered with the matrix according to the invention was found to be considerably improved. Finally the matrix also has the advantage that it can be manufactured in numerous variants depending on the respective production processes and with regard to their appearance, feel and taste. These advantages could not be foreseen.

According to the present invention the term “matrix” is defined as the entirety of supporting material and bioactive component, whereby the bioactive component may be homogeneously or heterogeneously dispersed in the supporting material or the supporting material may at least partially enclose the bioactive component as a coat; however, the bioactive component can also be applied to the supporting material. Also mixed forms of these variants are possible.

The term “hard” defines the state of aggregation of the claimed matrix in the sense of it not being soft and thus includes all states that are compatible and independent of the outer shape such as pellets, granulates, hard capsules etc. Thus soft gelatin capsules explicitly do not fall under this definition. However, amorphous, plastic types of hard consistency fulfil the requirements of a hard matrix.

The term “bioactive” is understood in the following as the effect of phospholipids, during or after release from the capsule, to develop a biological effect which usually applies to pharmaceutical preparations in the human and veterinary field in the area of absorption, on the transport path or at the target site in the living organism.

According to the present invention a matrix is preferred which contains between 5 and 90% by weight, in particular between 20 and 80% by weight based on the starting material of acetone-inisoluble phospholipid components, amounts between 40 and 70% by weight being particularly preferred. Phosphatidyl serine, phosphatidyl choline, phosphatidyl ethanolamine, phosphatidyl inositol, phosphatidyl glycerol, lyso compounds thereof and/or derivatives thereof are preferred acetone-insoluble components in the sense of the invention. In addition to the head group (i.e. serine, choline, inositol etc.), these compounds preferably contain one residue at each position sn-1 or sn-2 which is derived from a C₂-C₃₀ carboxylic acid, in particular a C₁₂-C₂₈ carboxylic acid bound to the hydroxy groups of the glycerol. The acidic residues can be linear or branched, saturated or monosaturated or polyunsaturated.

Particularly preferred residues are residues that are formed by the binding of acetic acid, butyric acid, caproic acid, caprylic acid, capric acid, lauric acid, myristic acid, arachidic acid, behenic acid, lignoceric acid, β-linolenic acid, eicosapentaenoic acid, erucic acid, nervonic acid, α- or β-eleostearic acid or parinaric acid. Residues are particularly preferred which are formed by the binding of palmitic acid, stearic acid, oleic acid, linoleic acid, α-linolenic acid, arachidonic acid or docosahexaenoic acid. The acidic residues bound to the OH groups of the glycerol that are still available can in this case be the same or different.

Furthermore, sphingophospholipids and thereof preferably sphingomyelin and derivatives thereof have turned out to be particularly suitable.

(Un)-modified carbohydrates and proteins, hydrophobic materials such as waxes, triglycerides, lipids and polymers or mineral components such as silicates and mixtures thereof have proven to be particularly suitable as “hard” supporting materials. The lipids chain be hydrogenated or have a special composition; the polymers can be pharmaceutical polymers and/or polymers suitable for foods. In this connection cereal products of maize, wheat, oats, rice etc. deserve particular mention which represent typical hard supporting materials as flakes or extrudates.

In order to take the respective matrix forms and applications into account, the invention envisages that in particular starch (derivatives), mono- and disaccharides and their sugar alcohols, glucose syrup, dextrins and hydrocolloids such as alginates, pectins, chitosan and cellulose (derivatives) are used as representatives of the carbohydrates. Particularly suitable representatives of proteins are plant, animal and microbial proteins such as zein, gluten, gelatin, casein, whey proteins and single-cell proteins, texturized proteins such is spun or extruded (soybean) protein isolate or mixtures thereof.

Each of the special representatives can of course be supplemented by other suitable supporting materials if required of which maltodextrins, sucrose, mono- and disaccharides and alcohols thereof, modified starches (e.g. esters and ethers), gum acacia, xanthan gum, gum arabic, carrageenan, furcelleran, agar, alginates, tragacanth and carboxymethyl cellulose are particularly recommended as carbohydrates.

Hydrogenated vegetable oils can also be used as hydrophobic materials as an addition to the preferred representatives and also natural oils such as palm oil, cotton seed oil, soybean oil, maize oil, palm kernel oil, babassu oil, sunflower oil and safflower oil can be used which can also be mixed with bee wax, petroleum-based paraffin wax, rice bran wax, castor wax, candellila wax, carnauba wax, shellac and microcrystalline wax.

Other recommended representatives of the lipids are tristearins, stearic acid and fats, and of course the phospholipids themselves can also be selected according to the prior art as a coat or a component thereof.

The range which can be covered by the supporting material is just as wide as the range for the proportion of bioactive component. Proportions of ≦95% by weight and in particular proportions between 30 and 80% by weight based on the total matrix weight have proven to be effective. The proportion of supporting material in the matrix is preferably ≧5% by weight, in particular ≧10% by weight, more preferably ≧20% by weight, even more preferably ≧40% by weight and most preferably >5.0% by weight and up to95% by weight, in particular up to 90% by weight, more preferably up to 70% by weight and even more preferably up to 60% by weight. In this manner the amounts of ingredient can be exactly adapted to the type of supporting material and to the respective application.

Although the present invention primarily concerns a stable and hard matrix which mainly contains phospholipids is a bioactive component, the matrix can of course also contain other bioactive substances such as amino acids, vitamins, polyphenols, carbohydrates, lipids, trace elements, mineral substances and suitable derivatives thereof. The essential amino acids are especially suitable and also for example creatine or other special amino acids such as theanine and derivatives thereof; fat-soluble vitamins such as those of the vitamin E family, the tocotrienols, phytostearins and other bioactive substances that accompany fats as well as representatives of the vitamin D series or vitamin C which deviate from the phospholipids can be used as representatives of the vitamins. Typical fish oil lipids have also proven to be suitable such as docosahexaenoic and eicosahexaenoic acid or in general omega-3 fatty acids in a triglyceride form and also conjugated linolenic acid. These other bioactive substances can be added to the supporting material, the bioactive component or both.

According to the invention substances are particularly suitable as supporting materials which enable a complete encapsulation as well as substances which provide a matrix with high stability and low shear stress.

An essential feature of the invention is among others the special diameter of the claimed matrix which differentiates it from the known larger soft gelatin capsules in addition to its hard state.

Within the claimed broad spectrum, diameters of the total matrix are regarded as preferred which are between 10 μm and 1000 μm and in particular between 50 and 500 μm.

As already mentioned, the claimed matrix is not limited to a special form and hence it can have a spherical, round or irregular shape. However, spherical or lens-shaped embodiments have proven to be particularly suitable but of course all other shape variants such as cylinders, cushions, amorphous states (e.g. flakes) and suchlike also come into consideration depending on the application and are always, of course, composed of the supporting material and the bioactive component.

Finally with regard to the matrix contents the invention preferably provides that this has a liquid consistency which then of course necessitates a rigid and hard coat.

Matrix variants in the form of a microcapsule have proven to be particularly suitable and are also encompassed by the invention.

In addition to the actual matrix, the present invention also claims their use in functional foods, special foods and dietary supplements where a delayed release of the bioactive component is particularly important. However, the retarding effect does not exclude the possibility that the complete matrix or components thereof (supporting material, bioactive component) can be attacked by gastric juices or be subject to chemical and/or enzymatic effects in the GI-tract. A preferred use for the claimed matrix is to prevent an elevated serum cholesterol level and (a)typical diabetes symptoms and also to strengthen mental fitness, exercise tolerance and physical and mental fitness.

All suitable methods of the prior art as well as similar or derived methods come into consideration for manufacturing the matrix according to the invention which additionally underlines the advantages of the invention.

Thus the claimed matrix represents a particularly suitable form of administration due to its special, possible features such as diameter, coating and capsule core since it can be produced in numerous forms and flavours and can therefore be readily added with high intrinsic stability to solid, semisolid and liquid foods.

Direct oral administration is of course the most suitable form of administration.

The following examples underline the advantages of the physiologically compatible phospholipid-containing, stable and hard matrix according to the invention.

### EXAMPLES

### Example 1

Microcapsule Containing 8% by Weight Phosphatidyl Serine

A 20% by weight solution of phosphatidyl serine (LeciPS® 20F from the Degussa BioActives GmbH) consisting of a mixture of triglycerides, phospholipids and glycolipids was encapsulated in a matrix with a natural vegetable fat using the known “spray technology”. The natural vegetable fat was characterized by the following features:

Melting point ca 55° C., peroxide number max. 2 meq 0/kg, acid number max. 1 mg KOH/g, iodine number max. 5 gl/100 g, saponification number 185-215 mg KOH/g, more than 94% of the natural acids (ca. 33% palmitic acid, ca. 60% stearic acid) were saturated.

The spherical matrix obtained in this manner in the form of microcapsules had an average total diameter of 100 to 250 μm and the following composition: 8% by weight phosphatidyl serine, 55% by weight vegetable fat and 37% by weight of a mixture of triglycerides, glycolipids and other phospholipids.

Stability of the Phospholipids:

Table 1 shows for the highly hydrolysis-sensitive phosphatidyl serine that embedding the phospholipids in the matrix according to the invention results in a stabilizing effect towards hydrolysis among others.

### Example 2

Microcapsule Containing 14% by Weight Phosphatidyl Choline

A 35% by weight solution of phosphatidyl choline (Epikuron® 135F from the Degussa BioActives GmbH) consisting of a mixture of triglycerides, phospholipids and glycolipids was encapsulated in a matrix with a natural vegetable fat using the known “spray technology”. The natural vegetable fat was characterized by the following features:

Melting point ca 55° C., peroxide number max. 2 meq 0/kg, acid number max. 1 mg KOH/g, iodine number max. 5 gl/100 g, saponification number 185-215 mg KOH/g, more than 94% of the natural acids (ca. 33% palmitic acid, ca. 60% stearic acid) were saturated.

The spherical matrix obtained in this manner in the form of microcapsules had an average total diameter of 100 to 250 μm and the following composition: 14% by weight phosphatidyl choline, 46% by weight vegetable fat and 40% by weight of a mixture of tiiglycerides lycolipids and other phospholipids.

Stability of the Phospholipids:

Table 1 shows for the highly hydrolysis-sensitive phosphatidyl choline that embedding the phospholipids in the matrix according to the invention results in a stabilizing effect towards hydrolysis among others.

### Example 3

Microcapsule Containing 50% by Weight Phosphatidyl Serine

A 90% phosphatidyl serine powder in the form of a lecithin concentrated from soya beans (LeciPS® 90 PN from the Degussa BioActives GmbH) was encapsulated in a matrix with a natural vegetable fat using the known “fluid-bed technology”. The natural vegetable fat was characterized by the following features:

Melting point ca 55° C., peroxide number max. 2 meq 0/kg, acid number max. 1 mg KOH/g, iodine number max. 5 gl/100 g, saponification number 185-215 mg KOH/g, more than 94% of the natural acids (ca. 33% palmitic acid, ca. 60% stearic acid) were saturated.

The spherical matrix obtained in this manner in the form of microcapsules had an average total diameter of 100 to 250 μm and the following composition: 5% by weight phosphlatidyl serine, 45% by weight vegetable fat and 5% by weight of other phospholipids.

Stability of the Phosplholipids:

Table 1 shows for the highly hydrolysis-sensitive phosphatidyl serine that embedding the phospholipids in the matrix according to the invention results in a stabilizing effect towards hydrolysis among others.

Table 1

The respective phospholipid-containing lecithin (examples 1 and 3: phosphatidyl serine; example 2: phosphatidyl choline) which was stored without matrix in an aqueous solution (pH 3.5; T=4° C.) served as a comparison.

The examples of the invention were carried out using phospholipid-containing microcapsules (examples 1 and 3: phosphatidyl serine; example 2: phosphatidyl choline) in which the microcapsules were stored in an acidic fruit juice (pH 3.5; t=4° C.) as an example of a typical functional food.

| | | Initial value | After 6 days | After 12 days | After 7 weeks |
|---|---|---|---|---|---|
| Example | Product and storage conditions | [%] | [%] | [%] | [%] |
| 1 | phosphatidyl serine (comparison) | 100 | 60 | 32 | 8 |
| | phosphatidyl serine (invention) | 100 | 98 | 96 | 90 |
| 2 | phosphatidyl choline (comparison) | 100 | 63 | 35 | 13 |
| | phosphatidyl choline (invention) | 100 | 98 | 95 | 91 |
| 3 | phosphatidyl serine (comparison) | 100 | 58 | 30 | 8 |
| | phosphatidyl serine (invention) | 100 | 97 | 94 | 91 |

1-14. (canceled)

15. A method for producing a physiologically compatible phospholipid-containing stable and hard matrix consisting of a supporting material and a bioactive component, the method comprising the steps of:
providing an acetone-insoluble phospholipid component as said bioactive component as a starting material;
providing the supporting material; and
preparing said matrix;
wherein said matrix has a total diameter between 0.1 μm and 5000 μm and contains ≧5% by weight, based on the starting material, of acetone-insoluble phospholipid components as the bioactive component.

16. The method as claimed in claim 15, wherein the matrix contains between 5 and 90% by weight, based on the starting material, of acetone-insoluble phospholipid components.

17. The method as claimed in claim 16, wherein the matrix contains between 20 and 80% by weight, based on the starting material, of acetone-insoluble phospholipid components.

18. The method as claimed in claim 17, wherein the matrix contains between 40 and 70% by weight, based on the starting material, of acetone-insoluble phospholipid components.

19. The method as claimed in claim 15, wherein the matrix contains (lyso)phosphatidyl serine, (lyso)phosphatidyl choline, (lyso)phosphatidyl ethanolamine, (lyso)phosphatidyl inositol, (lyso)phosphatidyl glycerol and/or derivatives thereof and/or sphingophospholipids, in particular sphingomyelin as acetone-insoluble components.

20. The method as claimed in claim 15, wherein the supporting material contains (un)modified carbohydrates and proteins, hydrophobic materials such as waxes, triglycerides, lipids and polymers or mineral components such as silicates and mixtures thereof.

21. The method as claimed in claim 20, wherein the carbohydrates are starch (derivatives), mono- and disaccharides and sugar alcohols thereof, glucose syrup, dextrins and hydrocolloids such as alginates, pectins, chitosan and cellulose (derivatives).

22. The method as claimed in claim 20, wherein the proteins are plant, animal or microbial proteins such as zein, gluten, gelatin, casein or whey proteins, soybean protein as well as single-cell proteins, texturized proteins or mixtures thereof.

23. The method as claimed in claim 20, wherein the proportion of supporting material is ≦95% by weight, based on the total weight of the matrix.

24. The method as claimed in claim 23, wherein the proportion of supporting material is between 30 and 80% by weight, based on the total weight of the matrix.

25. The method as claimed in claim 15, wherein the matrix contains additional bioactive substances such as amino acids, vitamins, polyphenols, carbohydrates, lipids, trace elements, mineral substances and suitable derivatives thereof.

26. The method as claimed in claim 15, wherein the total matrix has a diameter between 10 μm and 1000 μm.

27. The method as claimed in claim 26, wherein the total matrix has a diameter between 50 and 500μm.

28. The method as claimed in claim 15, wherein the matrix is spherical.

29. The method as claimed in claim 15, wherein the matrix is lens-shaped.

30. The method as claimed in claim 15, wherein the matrix has liquid contents.

31. The method as claimed in claim 15, wherein the matrix is a microcapsule.

32. Functional foods, special foods and dietary supplements comprising the matrix of claim 15.

33. Foods and dietary supplements with delayed release comprising the matrix of claim 15.

34. Method of preventing elevated serum cholesterol levels and diabetes symptoms, strengthening mental fitness, exercise tolerance and fitness by administering the matrix of claim 15.
